(19) United States
(12) Patent Application Publication
(10) Pub. No.: US 2023/0342547 A1
(43) Pub. Date: Oct. 26, 2023

(54) System and Method for Natural Language Translation and Reconciliation of Colloquial Chemical Terminology

(71) Applicant: Nima Veiseh, Grand Haven, MI (US)

(72) Inventor: Nima Veiseh, Grand Haven, MI (US)

(21) Appl. No.: 18/302,505

(22) Filed: Apr. 18, 2023

Related U.S. Application Data
(60) Provisional application No. 63/333,950, filed on Apr. 22, 2022.

(51) Int. Cl. G06F 40/242; G06F 40/30

(52) U.S. Cl. CPC G06F 40/242; G06F 40/30

(57) ABSTRACT

Disclosed is a system and method for natural language translation and reconciliation of colloquial chemical terminology.

The current application claims a priority to the U.S. provisional patent application Ser. No. 63/333,950 filed on Apr. 22, 2022.

### FIELD OF THE INVENTION

The present invention relates generally to a method for a data structure. More specifically, the present invention is a method for a data structure containing meta-data.

### BACKGROUND OF THE INVENTION

It is a non-obvious problem because of the institutional disconnect between academic, regulatory, and government content combined with traditional day-to-day communication. It is essential to be able to reconcile the different terminology that may be associated with a specific molecule. This is especially and critically important in the pharmaceutical industry and its ancillary participants because there are no current tool kits are used for uniting the lineage of knowledge and most recent advisory issues currently. Thus, it is imperative to invent a tool that allows the reconciliation of these terminologies by creating a self-documenting algorithmically driven data structure that can perform this necessary and potentially life-saving colloquial reconciliation across disconnected documents.

### BRIEF DESCRIPTION OF THE DRAWINGS

FIG. 1 is a diagram of the present invention.

### DETAIL DESCRIPTIONS OF THE INVENTION

All illustrations of the drawings are for the purpose of describing selected versions of the present invention and are not intended to limit the scope of the present invention.

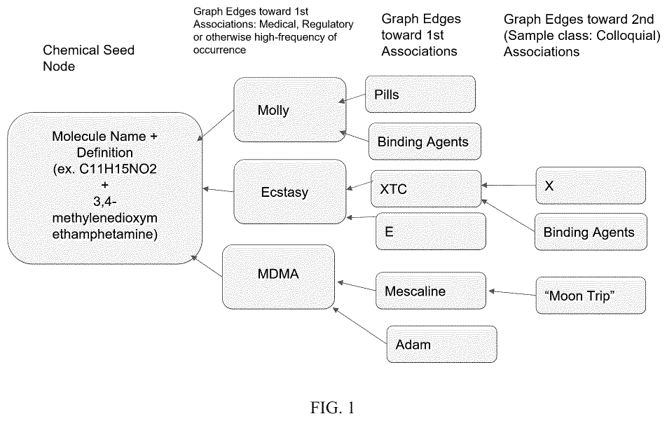

As can be seen in FIG. 1, the non-obvious nature of the present invention involves focusing on the unique characteristics of the specific type of content pool we are addressing in that each term can be tied to a digitally tracked colloquial lineage to a standardized molecule. This is unlike other colloquial reconciliation mechanisms because both: Dictionaries can exist differently between languages, and a single language can have multiple dictionaries. For example, the English language has multiple dictionaries, e.g., Oxford or Webster.

The present inventor has invented a Two-part data structure where the base of the structure (initialization node) is the seed. This seed contains a meta-data interaction definition of a molecule that is exogenous and unaffected by linguistic translations because it's anchored in a language-agnostic chemical definition.

From this seed definition that is non-linguistic, the present inventor then activates a combing process for navigating a pool of documents and creating an interactive, nested Ranking system that utilizes breadth-first search in order to associate and identify terms immediately associated with the molecule. Propagating a colloquial crawler to then find subsequent terms that may have a further geometric distance in the dataset from the seed molecule and its most immediate definition in the particular language or alphabet.

The documents in the “document pool” can include by not limited to: academic publications, public regulator announcements, conversation transcripts, blogs, forums, social media platforms, online menus, scanned documents, metadata, computer code, etc, and can span any existing, or yet to be developed symbolic language.

The outcome of all of this is a deterministic process that identifies, ranks, and consolidates all of the necessarily associated terminologies that can then enable commercial and academic explorers to have a more comprehensive and detailed view of the literary ecosystem that contains the terminology associated with the chemical.

The outcome is also a weighted average nodal representation which can then be used to calculate colloquial sentiment surrounding the chemical in real time. The present invention also works to help to overcome social inequity and racial barriers given the necessity and use of slang in identifying and naming these molecules. This is historical because of the disjointed regulatory space both at a national and international level with regards to the lack of agreement with regards to access to these substances. By creating a methodology to reconcile these terminologies we can jumpstart research that may have slowed due to the fragmented environment. It may be possible to enhance health outcomes and accelerate research processes and workflows by creating a single point of understanding with regards to how we as biological and social creatures interact, and report their interactions, with these molecules.

The prior art for the present invention includes the following:
1. U.S. Pat. No. 8,990,066B2
2. U.S. Pat. No. 8,631,126B2
3. WO2006091528A3
4. WO2009110991A2
5. U.S. Pat. No. 10,909,328B2
6. US20160343004A1
7. U.S. Pat. No. 8,838,633B2
8. “Markets With Memory”, by Nima Veiseh, 2019.ISBN: 9781700165121

Although the invention has been explained in relation to its preferred embodiment, it is to be understood that many other possible modifications and variations can be made without departing from the spirit and scope of the invention.

What is claimed is:

1. A method of automatically generating a colloquial complete definitions table for chemical compounds, the method comprising the steps of:
(A) providing an algorithmically technology that incorporates both the chemical structure and the textual colloquial definitions;
(B) reconciling the ecosystem literature for chemical and active pharmaceutical ingredients as well as cannabinoids, psychedelics, and other types of treatments;
(C) executing an enhanced capability for performing sentiment analysis relating to, but not limited to, pharmaceutical and non-pharmaceutical; and
(D) executing an augmented NLP algorithm for reconciling scientific definitions, and utilizing, but is not limited to, a graph DB structure, wherein implementation can be activated through SQL, NoSQL, and other relational and non-relational data structures.
